(19) United States
(12) Patent Application Publication
(10) Pub. No.: US 2006/0030782 A1
(43) Pub. Date: Feb. 9, 2006

(54) Heart disease detection patch

(76) Inventor: Adnan Shennib, Dublin, CA (US)

Correspondence Address: GLENN PATENT GROUP, 3475 EDISON WAY, SUITE L, MENLO PARK, CA, 94025, US

(21) Appl. No.: 10/913,586

(22) Filed: Aug. 5, 2004

(51) Int. Cl. A61B 5/0402

(52) U.S. Cl. 600/509

(57) ABSTRACT

The invention provides a disposable sensor patch for the non-invasive detection of heart disease. The patch is placed on a person's chest area for automatic analysis of ECG. The heart condition is indicated via an indicator integrated within the patch. The patch is inexpensive and simple for self-administration. In one embodiment, the status of the heart is indicated via multiple LEDs. The detection and indication typically occurs, within 24 hours or sooner if a condition is readily identifiable. The patch is thin, flexible, and incorporates a battery, ECG amplifier, and a processor for analyzing ECG waveform and indicating the heart condition. A software algorithm searches for a cardiac abnormality such as arrhythmia, bradycardia, tachycardia, fibrillation, mycocardial infarction, ischemia, long-QT syndrome, blocks, late potentials, and premature contractions. In another embodiment, results and relevant ECG data are stored in memory for later retrieval.

### CROSS REFERENCE TO RELATED APPLICATIONS

This application is related to co-pending patent application entitled Emergency Heart Sensor Patch, filed jointly with this application, which application is incorporated herein in its entirety by the reference thereto.

### BACKGROUND OF THE INVENTION

1. Technical Field

The invention relates to electrocardiogram (ECG). More particularly, this invention relates to non-invasive detection of heart disease.

2. Description of the Prior Art

Cardiovascular diseases are pervasive, contributing to over 2.4 million deaths annually in the United States alone. Although misconceived as primarily an old Wan's disease, cardiovascular disease causes about a death a minute among females and is the third most common cause of death for children under the age of fifteen. Estimates of heart attach range from 1.2 to 1.5 million with 700,000 new cases reported annually. About 42 percent of heart attacks result in death and about 80 percent of coronary heart disease mortality in people under the age of 65 occurs during the first attack.

Patients suffering from heart disease often have no symptoms until a heart attack develops. Symptoms of heart disease include discomfort in the chest, shortness of breath, nausea, light-headedness, and palpitations. Delay in recognition and treatment of heart disease leads to more damage to the heart and other vital organs, such as the brain. Delayed recognition and treatment also leads to higher cost of hospitalization and lower quality of life for the survivors.

For a variety of reasons, including lack of symptoms, lack of awareness, rising health care costs, or simply the hassles of seeking qualified diagnosis, most cardiac related problems are undiagnosed, particularly at the early stage. Conventional detection methods and instruments are problematic for early detection. Pulse detection, a rudimentary indicator of heart activity, is inadequate for assessing most heart diseases. Non-invasive sensing of surface potentials of cardiac electrical activity, i.e. the electrocardiogram (ECG), remains one of the most reliable and effective method for proper diagnosis of cardiac function.

Conventional ECG methods involve attaching electrodes to the body, mostly on the chest near the heart, and connecting electrode wires (cables) to an electronic instrument having a monitor that displays the ECG waveform. Medical personnel skilled in ECG interpretation can readily recognize heart abnormalities by visual observation. ECG interpretations can also be automated by a microprocessor (processor) incorporated within the ECG instrument. However, the cost, bulk, and complexity of standard ECG instruments render its application impractical outside of medical settings.

Holter monitors are specialized instruments for long term ECG monitoring at home; for example see U.S. Pat. No. 6,456,872 to Faisandier. These instruments typically use 5 or more ECG electrodes attached to the chest at one end connected to a portable device at the other end. The device is worn or strapped to the body and records ECG signals in its memory. Holter monitors may also incorporate an alarm to warn the patient of an adverse cardiac event. After 24 or 48 hours of monitoring, the Holter monitor is typically returned to the clinic, where the recorded ECG data are downloaded for review, record keeping, and for further analysis, if necessary. Trans-telephonic data transmission of ECG data is also widely employed for individuals who require longer term or daily monitoring of their ECG. However, Holter monitors and other portable ECG instruments are also relatively expensive and cumbersome, and thus are typically offered only to select patients as prescribed by a physician.

Cardiac event recorders are hand-held ECG instruments with integrated electronics for instant, momentary self-application of the device on the chest whenever a cardiac event is suspected, i.e. heart palpitation, dizziness, chest pain, etc. Event recorders typically have limited memory to record only a few minutes of ECG data.

There are also a variety of non-medical consumer-oriented heart and pulse monitors available for wellness and fitness applications. These devices are offered in the form of a wristwatch, or may be belt-worn or pocket-worn. They may have built-in electrodes or be provided with cable-connected electrodes for sensing and computing certain ECG parameters, such as instantaneous and average heart rate. Although considerably less expensive than Holter monitors and ECG event recorders, these monitors offer only limited medical diagnostic ability and, thus, are not suitable for detecting most cardiac abnormalities.

Detection of cardiac abnormalities from ECG analysis is possible and highly desirable.

For example, myocardial ischemia, considered to be the most common trigger of fatal arrhythmias, can be detected from ECG analysis. Non-ischemic abnormalities, such as long-QT syndrome, are also detectable. Unfortunately, barriers to proper diagnosis continue to exist with conventional ECG methods and instruments.

Access to qualified medical care for cardiac screening or diagnosis presents another problem for most people with potential heart conditions. Many cardiac care centers are overwhelmed and the wait times to see a heart specialist can be several months, particularly for cases presumed non-urgent. Even for individuals suspected of having an urgent condition, the wait can be several weeks. It would be very desirable to perform an effective cardiac test as soon as possible, particularly for those suffering a heart abnormality with a potentially fatal outcome. Prior art instruments and methods discussed above, and other discussed below, fall short in achieving these objectives.

U.S. patent application Ser. No. 2003/0069510 to Semler discloses a disposable vital signs monitor in the form of a patch that is a “flexible, nominally flat planer form having integral gel electrodes, a sticky-back rear surface, an internal flex circuit capable of sensing, recording, and play out several minutes of the most recently acquired ECG waveform data and a front surface that includes an output port preferably having one or more snap connectors compatible with lead harness . . . .” The playback and analysis is presumably performed in a medical setting under the supervision of skilled medical personnel. In another embodiment of Semler's invention, the monitor is remotely controlled by telemetry and is capable of delivering pacer or defibrillation pulses to the patient. Although inexpensive as a disposable event recorder, it provides no integrated analysis or indication of the heart condition. Therefore, Semler's invention has limited application for assessment of a person's heart condition.

U.S. Pat. No. 6,112,116 to Fischell et al., U.S. Pat. No. 5,313,953 to Yomtov et al., U.S. Pat. No. 6,501,983 to Natarajan et al., U.S. Pat. No. 5,135,004 to Adams et al., and U.S. Pat. No. 6,272,379 to Fischell et al, disclose implant devices and methods that detect various cardiac events, such as myocardial infarction (MI) and ischemia. The complexity and invasive nature of these implants render them impractical for screening applications, and they thus are limited to high-risk individuals who are already diagnosed with heart disease.

U.S. Pat. No. 6,609,023 to Fischell et al. discloses a system for detection of a cardiac event. The system incorporates an alarm system which may be internal or external to the device. The embodiments disclosed by Fischell's '023 patent concern an implant device which measures the ECG internal to the human body. Fischell also discloses, very briefly, an external embodiment, without any details of the actual configuration, presumably a standard cardiac event recorder or ECG instrument, as discussed above.

U.S. patent application Ser. No. 2003/0083559 to Thompson discloses a peripheral monitor patch for attachment to a patient including high capacity memory for storage and later retrieval of the sensed ECG data. The patch comprises non-contact electrodes. The patch neither provides diagnostic capability nor indication of heart condition.

U.S. Pat. No. 6,690,959 to Thompson discloses a smart patch with nano-spikes for improving the electrode-skin contact. Similarly, the '959 patent does not teach a built-in diagnostic and indicator means to the wearer or others.

Kagan et al. in U.S. Pat. No. 5,443,072 disclose a disposable blood flow monitor which is adhered directly to the skin above the vessel to be monitored. Kagan's invention does not concern analysis or indication of heart function.

Hagen et al. in U.S. Pat. No. 6,572,636 disclose a pulse sensing patch with an indicator for displaying a visually recognizable pattern of detected pulses. As discussed above, pulse detection provides inadequate diagnosis in most heart abnormality case.

It would be advantageous to provide an inexpensive, non-invasive heart condition detector for ambulatory home use without the involvement of specialized medical personnel or conventional ECG instruments.

It would also be advantageous to provide an automatic heart function detector that is simple to self-administer or be administered by a layperson assisting a person being examined.

It would also be advantageous to provide an inexpensive method to detect and indicate a potential heart abnormality.

It would also be advantageous to provide an inexpensive device for early detection of a cardiac condition and to indicate the need for specialized cardiac care.

It would also be advantageous to provide a readily accessible Over-the-counter heart screening device that is inexpensive and user friendly.

### SUMMARY OF THE INVENTION

The invention provides a disposable sensor patch for the non-invasive detection of a heart condition. The patch is placed on a person's chest area for sensing and analyzing the surface electrocardiogram (ECG). The smart patch automatically obtains and analyzes ECG signals and searches for abnormalities. A heart condition is indicated via an indicator integrated into the patch. The smart patch is inexpensive, simple to use, and suitable for self-administration. The patch is activated automatically upon its removal from the package and placement on the chest. In one embodiment, the status of the heart is indicated via multiple LEDs. The detection and indication typically occurs within 24 hours, or sooner if a condition is readily identifiable.

The smart cardiac patch is thin, flexible, and incorporates a battery, indicator, electrodes, ECG amplifier, and a processor for analyzing ECG waveform and detecting and indicating the heart condition. The software algorithm executed by the built-in processor searches for a cardiac abnormality, such as arrhythmia, bradycardia, tachycardia, fibrillation, mycocardial infarction, ischemia, long-QT syndrome, blocks, late potentials, and premature contractions.

The disposable patch is designed to be inexpensive and readily accessible, to encourage early identification of possible cardiac disease in everyday settings and without resorting to specialized medical care. At least two levels of indication are provided to the user. For example, a normal condition vs. a risk condition. Other embodiments include multiple risk level assessment, such as normal, low risk, high risk and urgent conditions. An LCD is particularly appropriate to indicate multiple risk assessment to the user. The indication may be partially or fully deferred to a physician for verification and proper disclosure of a patient's condition.

In another embodiment, memory is provided to record analysis results and relevant ECG data. Stored results and related ECG data are subsequently retrieved by an interrogation device, e.g. in a clinical setup. This feature provides a record of transient cardiac events, which, if not recorded, are often illusive for medical personnel to detect and document subsequently.

Ischemic disease detection is of particular interest for the invention. Other non-ischemic diseases, congenital or acquired, are also of interest. The disposable patch may be offered in a generic form or targeted for specific age, sex, test condition or disease groups.

### BRIEF DESCRIPTION OF THE DRAWINGS

FIG. 1 is a view of the heart abnormality test patch placed on the chest of a person;

FIGS. 2a-2c show various ECG patterns: FIG. 2a shows (a) normal ECG, FIG. 2b shows ischemic condition ECG with characteristic ST-segment depression and FIG. 2c shows myocardial infarction (heart attack);

FIG. 3 is a top view of the cardiac abnormality detector patch showing four electrodes, flexible circuit, battery, and other major components;

FIG. 4 is a cross section view of the patch of FIG. 3, showing the various layers with thickness exaggerated for clarity;

FIG. 5 shows a two-electrode band-shaped embodiment;

FIG. 6 shows a rectangular embodiment of the heart test patch with four electrodes and an LCD indicator;

FIG. 7 shows a C-shaped cardiac test patch for implementing leads I-III and V1-V5;

FIG. 8 shows the C-patch embodiment of FIG. 7 placed on the chest and encompassing the left breast of a female;

FIG. 9 shows a cardiac test patch embodiment for implementing standard five-electrode two-channel Holter monitor configuration;

FIG. 10 shows the two-electrode band embodiment of FIG. 5 placed on the chest area near the heart;

FIG. 11 shows the heart test patch with EASI lead system configuration; and

FIG. 12 shows optical transmission of ECG data, recorded by the heart abnormality detection patch, to an external device.

### DETAILED DESCRIPTION OF THE INVENTION

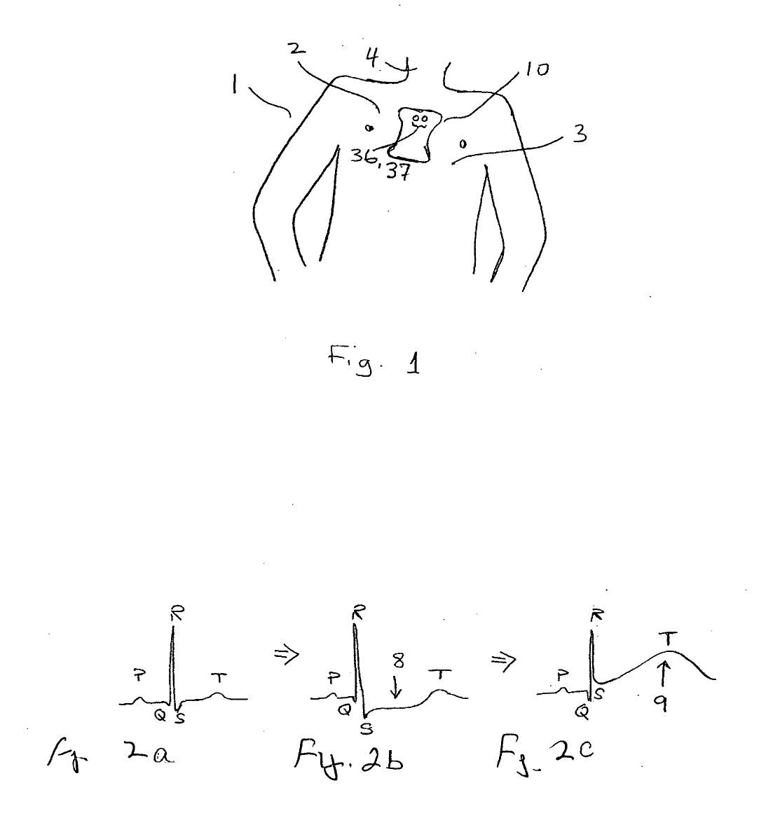

The invention, shown in various embodiments of FIGS. 1 and FIGS. 3-12 is a disposable non-invasive patch for detection and indication of heart abnormality. The patch 10 is thin, flat, and flexible for placement on the chest area 2 of a person 1 whose heart is being examined for possible abnormality. The sensor patch relies on a surface electrocardiogram (ECG) for detecting and analyzing non-invasively the electrical activity of the heart and indicating the results through an indicator integrated into the patch. The smart patch is fully self-contained and self-powered. The patch analyzes the ECG for an extended period of time, e.g. 24 to 48 hours or more, depending on the application. Patterns of ECG abnormalities are detected and the risk level is indicated to the user wearing the device. The electronic sensor patch is designed for inexpensive over the counter availability and primarily for self-administration.

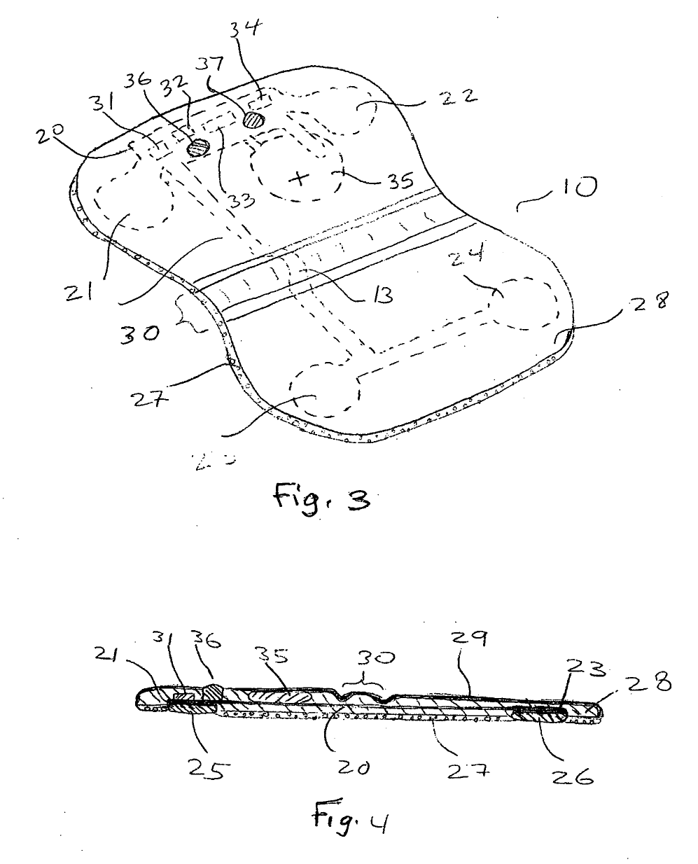

Referring to the embodiment of FIGS. 3 and 4, the sensor patch 10 comprises four ECG electrodes 21, 22, 23 and 24, an ECG amplifier 31, a processor 33, and a battery 35. The processor 33 is typically a digital signal processor for performing numerical computation from data obtained from an analog-to-digital converter 32. The sensor patch 10 also incorporates a memory 34, referring generally here to all types of electronic memory for storage of program data and acquired ECG data, if so desired.

The electronic assembly of the patch is formed of a flexible circuit substrate 20 with trace extensions to the electrodes 21, 22, 23, 24 and to the battery 35. Conductive gel 25, 26 covers the electrodes 21, 22, respectively, as well as other electrodes not shown in the view of FIG. 3. The conductive gel 25 and 26 contacts the person's skin directly to conduct surface ECG potentials to the electrodes and to the ECG amplifier 31. The electrodes may be pre-gelled as shown or alternately made for dry contact (not shown) with electrodes directly contacting the skin. A non-conductive pad 27 provides skin contact, preferably comprising a gel, i.e. Hydrogel, or an adhesive material for adhering the patch 10 to the skin. The pad 27 may be made of soft low-durometer rubber or elastomeric material. The patch 10 also comprises a thin substrate 28 for providing structural support. The substrate 28 is made of soft flexible sheath material, such as polyurethane, cloth or made from the same pad material. The thickness of the patch device 10 (not shown to scale for clarity) is preferably in the range of 1.5 and 2.5 mm, but preferably no more than 3.5 mm. A groove area 30 and trace loop 13 provide additional flexibility and folding area for the patch 10 while it is stored in its package.

In the embodiments of FIGS. 3, 4, and 6, the smart heart monitor patch 10 comprises four ECG electrodes for placement on the chest area as shown in FIG. 1. The electrodes are arranged to provide a modified three-lead configuration with the electrodes 21, 22, 23, 24 representing right arm (RA), left arm (LA), right leg (RL), and left leg (LL) leads in standard ECG instrumentation. This configuration results in standard, direct lead measurements Lead-I, Lead-II, Lead-III, as well as augmented leads aVR, aVL, and aVF.

The detection of cardiac abnormalities involves a wide range analysis from heart rate measurements to subtle waveform extraction and pattern recognition. For example, a heart rate exceeding 160 beats per minute (BPM) at rest readily indicates a tachycardia condition which may evolve to fibrillation and sudden cardiac death. A heart rate of 45 BPM or below indicates a low rate or bradycardia condition.

A person may be experiencing an abnormality that can lead to a heart attack, but currently exhibit a heart rate well within the normal range. Therefore, through the analysis of the ECG waveform, a serious condition can be revealed. The smart patch automatically provides analysis of the ECG waveform and indicates the abnormality, particularly those normally leading to fatal heart attacks.

FIGS. 2(a-c) show typical ECG patterns of ECG from normal and ischemia to a heart attack caused by a myocardial infarction (MI). Briefly described here, when the blood supply is reduced due to coronary heart disease, the oxygen supply to the heart muscle is reduced and the condition is referred to as ischemia. Prolonged or severe ischemia may have symptoms of chest pain. However, many patients do not experience any pain or discomfort. Thus, the asymptomatic ischemia is referred to as silent ischemia. ECG analysis is more sensitive than patients'symptoms for detecting myocardial ischemia and other conditions because 80% to 90% of ECG-detected episodes are clinically silent.

FIG. 2a shows, for reference purposes, a normal ECG consisting of a P wave, a QRS complex, and A T-wave. FIG. 2b shows a typical ischemic condition characterized by a depression (arrow 8) of the ST segment when compared to the normal baseline. Generally, the magnitude of the depression is proportional to the severity of the ischemic condition. FIG. 2c shows an early sign of MI indicated by a sharp increase in the amplitude and width of the T-wave (arrow 9). As MI progresses, the T-wave generally broadens further with elevation of the ST-segment indicating the likely occurrence of transmural injury. These patterns and others are well known in the field of cardiovascular disease and electrophysiology and provide reliable diagnosis of the heart condition.

Real-time ECG analysis in the invention is performed by the processor 33. Various cardiac abnormalities can be detected by comparing the characteristics of sensed ECG with predetermined limits and patterns. Furthermore, minor shifts in certain key segments, such the ST-segment and QRS width, can be detected to indicate possible abnormalities.

The detection of a heart condition is indicated by an indicator 36. In the embodiment shown in FIGS. 1 and 3, two light emitting diode (LED) indicators 36 and 37 are provided in two different colors. For example, a green LED light indicates a safe heart condition, while a red LED light indicates a risk condition. The LEDs can also be used to indicate general heart activity during the collection of ECG data and prior to determining the heart condition. For example, one or two of the LEDs can be flashing in synchrony with QRS pulses immediately upon placement of the smart patch on the chest and upon the detection of ECG signals. After 24 or 48 hours of sensing and analysis, either the green or red LED is activated depending on the results. A serious cardiac condition may be indicated promptly and well before 24 hours upon collecting sufficient data to verify the condition. For example, it may take five minutes or less to indicate an acute myocardial infraction. In the preferred embodiments, at least 90 seconds of analysis is required.

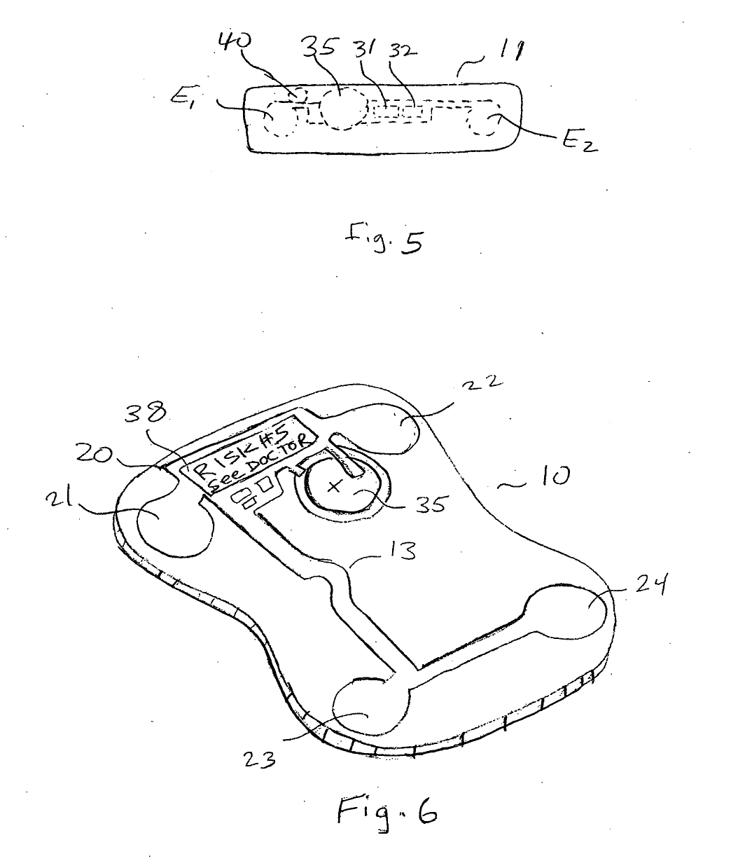

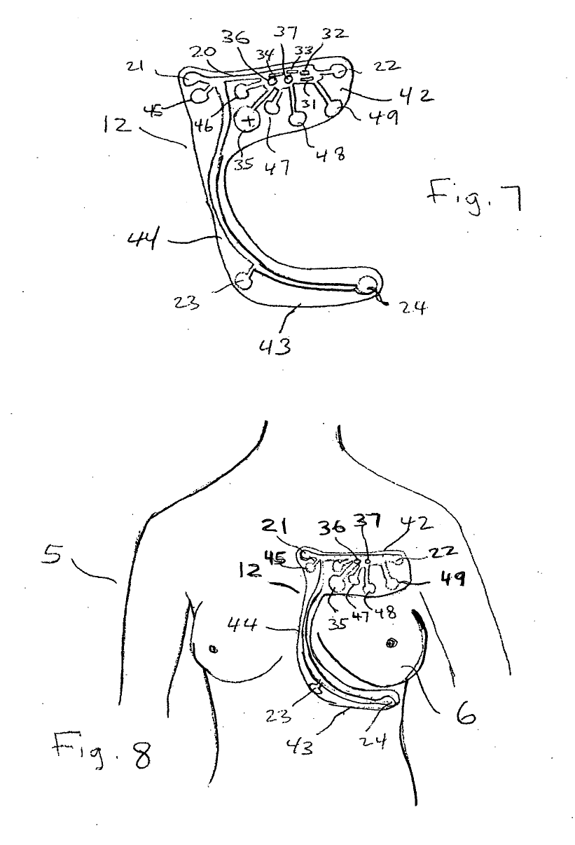

Other possible indicators include audible transducers, such as a buzzer (not shown) or a speaker (not shown; and other visual indicator types, such as a liquid crystal display (LCD) 38 as shown in FIG. 6. Electrochemical indictors (color strips) are also envisioned. The advantage of an LCD or multi-color indicator is the ability to indicate different levels of conditions such as “normal function,” “see doctor,” etc. A coded risk i.e. Risk #5 may also be displayed by an LCD for interpretation by a medical specialist or through instructions supplied with the disposable patch. An LCD indicator can also spell out the condition to communicate accurately the detected condition. A key feature of the invention in the preferred embodiment is integrating in a single low cost disposable patch the combination of ECG analysis and heart condition indication.

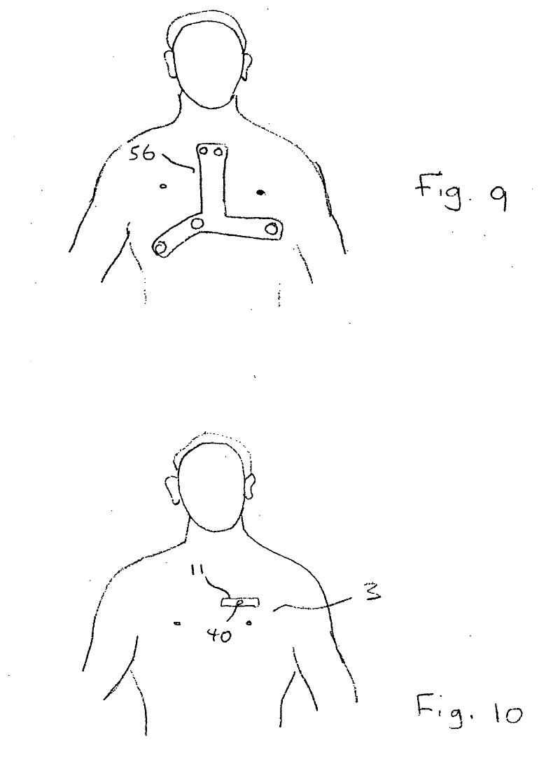

FIG. 7 shows a nine-electrode patch 12 arranged in a “C” configuration. The electrodes are arranged for modified twelve-lead system, excluding the V6 lead. This and other multi-lead configurations provide multi-axis or vectorcardiograph capability for improved diagnostics. The electrodes 21, 22, 23, 24 offer bipolar frontal plane ECG (lead-I, II, and III) while the electrodes 45, 46, 47, 48, and 49 offer unipolar precordial ECG, generally representing the horizontal plane, for leads V1, V2, V3, V4, and V5, respectively. The “C” patch encompasses the left breast 6 having an upper segment 42, lower segment 43, and sternum segment 44. The “C” patch 12 is particularly suitable for fitting on a female 5 as shown in FIG. 8.

FIG. 9 shows a five-electrode embodiment 56 with electrodes arranged in a similar manner as a two-channel Holter monitor. Other Holter monitor and event recorder electrode configurations are possible (not shown).

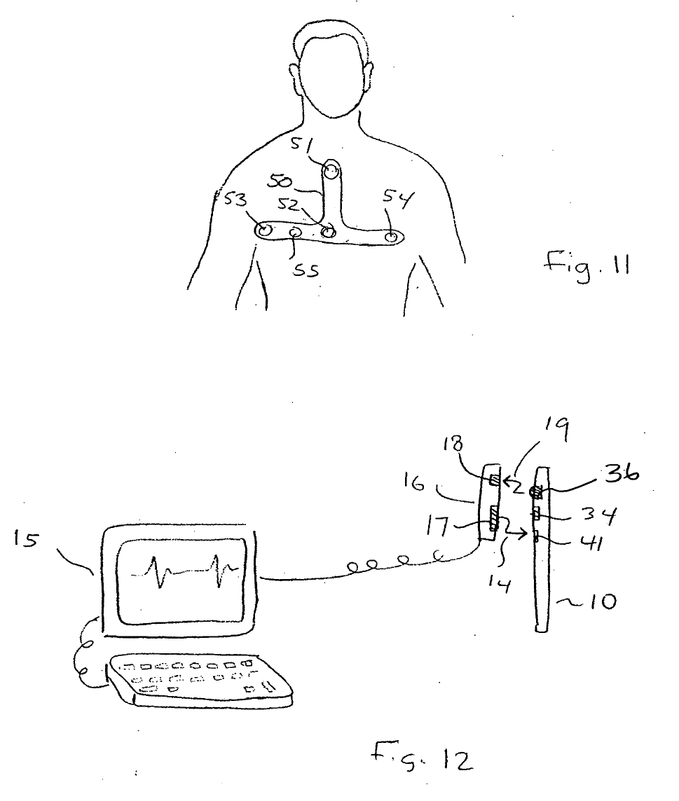

FIG. 10 shows a compact band-shaped patch 11 with a two-electrode embodiment for sensing surface ECG on the heart area 3 of the chest. A multi-color LED 40 is used to indicate heart activity and condition.

Other lead configurations have been developed to minimize the number of electrodes from which standard ECG leads can be derived through computations. FIG. 11 shows the EASI™ lead configuration whereby five electrodes as used to derive a twelve lead ECG. The EASI patch 50 uses the electrodes 51, 52, 53, 54 and 55, referred to as the S, E, I, A and Ground, respectively. EASI leads are transformed to a standard twelve-electrode configuration by the EASI algorithm, which is executed by the processor 33.

These and other electrode configurations are possible, as will become obvious to those skilled in the art of ECG measurements. Because the electrodes are integrated within the patch of the invention, motion artifact is significantly reduced when compared to standard ECG with separate electrodes and cabling. Furthermore, the integrated patch allows for inconspicuous, convenient ambulatory application.

Although ischemic disease detection is of particular interest, other non-ischemic diseases, congenital or acquired, are also of interest. In other embodiments of the invention, detection of a specific disease or condition may be provided to deal with particular abnormalities. For example, certain hereditary abnormalities are only common in certain groups or countries, such as Brugada sign in Southeast Asia, a condition associated with sudden arrhythmia death (SAD). Other Particular heart conditions can be detected only at rest, while others only occur during exercise. Certain cases of sudden infant death are attributed to fatal arrhythmia during sleep. Other applications include the detection of drug-induced arrhythmia, whereby its detection assists the physician in suggesting an alternative medication. These and other abnormalities can be easily investigated and indicated by the patch, which may be offered with generic detection algorithms or targeted for a specific abnormality, age and sex group, rest condition, or disease group. Although suitable for over the counter availability, the smart patch is equally applicable for prescription by a physician investigating a possible abnormality.

Various filtering methods are known in the field of signal processing and particularly pertaining to ECG signals. For example, notch filters are effective in removing 60-Hz noise present in the environment. To minimize electromagnetic interference, a metal foil 29 (FIG. 4) is provided over the patch, entirely, or selectively over components sensitive to the interference.

Signal processing is particularly applicable for performing signal averaging to enhance certain details of the sensed ECG. Signal-averaged ECG involves the averaging of a large number of ECG periods, particularly QRS complexes, to enhance the detection of small fluctuations. For example, late potentials present in QRS complexes generally indicate increased risk of sudden cardiac death. The detection, for example, of late potentials by the processor 33 is useful for screening patients prone to this and other high-risk conditions.

In another embodiment, a memory 34 is provided for automatic recording of abnormal ECG events. This feature provides a record of transient cardiac events which may become illusive for medical personnel to detect and document subsequently. The recorded ECG data are later retrieved by an interrogation device 15 (FIG. 12) in the clinic. The transmission of data preferably uses existing components to reduce cost and complexity of the disposable patch. For example, FIG. 12 (shown not to scale) shows the optical transmission 19 of ECG data using the LED indicator 36 incorporated within the disposable patch 10. In this embodiment, ECG data are transmitted from the LED indicator 36 to an optical receiver 18 incorporated in the interrogation interface 16 of the external interrogation device 15. The activation of the data transmission is preferably automatic. For example, a magnetic field 14 from a magnet 17 within the interface 16 triggers an activation sensor 41, i.e. a reed-switch, to initiate the ECG data transmission. Activation can also be by manual means, such as by pressing an electromechanical switch (not shown) incorporated onto the flexible substrate 20.

The wireless transmission of heart condition results and abnormal ECG may be accomplished in numerous ways and methods known in the field of medical devices and wireless data transmission. This includes optical means as shown above, or radio frequency (RF), magnetic, ultrasonic, and acoustic transmission. Inductive coupling through a coil (not shown) can also be used to transmit data, as well as for powering the patch externally during the transmission.

Proper adhesion to the skin is important for securing the patch to the person during the automatic examination of the heart. Furthermore, proper electrode-skin contact throughout device operation is necessary for obtaining an adequate ECG signal-to-noise-ratio. Proper electrode-skin contact can be determined automatically indirectly by measuring the impedance between adjacent electrodes. Normal electrode-electrode impedance for closely positioned electrodes is generally well under 10 k-ohms, depending on the condition of the skin and the distance between the electrodes. Measurement and detection of electrode-electrode impedance can also be used to activate the patch device 10 automatically upon its placement on the skin. Automatic activation can also be accomplished during the removal of the patch device 10 from its package, i.e. a pouch. For example, by incorporating open-circuit and/or short-circuit conditions between the electrodes within the package. These circuit conditions are altered during the removal of the patch device 10 from the package triggering the activation of the device. These and other automatic activation means and methods will be readily recognized by those skilled in the art of electronics and medical device packaging.

Although the invention is described herein with reference to the preferred embodiment, one skilled in the art will readily appreciate that other applications may be substituted for those set forth herein without departing from the spirit and scope of the present invention. Accordingly, the invention should only be limited by the Claims included below.

1. A patch for non-invasive detection of a heart condition, comprising:
means for adhering said patch to a person's chest, said person is being evaluated for a possible heart abnormality;
at least two electrodes for contacting said person's skin surface, said electrodes receiving the surface potential ECG signals;
an amplifier for amplifying said ECG signals from said electrodes;
a processor for performing analysis of said amplified ECG signals; an indicator;
a flexible substrate incorporating said amplifier, said processor, said electrodes, and said indicator; and
means for detecting a heart abnormality after analyzing said ECG signals by said processor and indicating a detected heart condition via said indicator.

2. The patch of claim 1, further comprising:
a battery for powering said patch.

3. The patch of claim 1, wherein said processor analyzes said ECG signals for at least 90 seconds.

4. The patch of claim 1, wherein said patch has a thickness of less than 3.5 mm.

5. The patch of claim 1, wherein said patch is self applied.

6. The patch of claim 1, further comprising:
a flexible, electronic circuit for interconnecting electronic components within said patch to said electrodes.

7. The patch of claim 1, wherein said electrodes are configured to obtain any of Lead-I, Lead-II, Lead-III, V1-lead, V2-lead, V3-lead, V4-lead, V5-lead, and V6-lead ECG signals.

8. The patch of claim 1, wherein said patch is “C” shaped encompassing the person's left breast.

9. The patch of claim 1, comprising five electrodes and configured to obtain EASI leads.

10. The patch of claim 1, wherein said patch is rectangular shaped.

11. The patch of claim 1, further comprising:
a memory for storing ECG data.

12. The patch of claim 11, further comprising:
means for transmitting ECG data stored in said memory to an external device.

13. The patch of device of claim 12, said means for transmitting ECG data comprising a transmitter element incorporated within said patch comprising any of an optical element, an RF element, an induction element and an electromagnetic element.

14. The patch of claim 12, further comprising:
means for activating transmission of ECG data.

15. The patch of claim 1, wherein said indicator comprises an audio transducer.

16. The patch of claim 1, said indicator comprising at least one visual indicator, comprising any of, a light emitting diode (LED), a color strip element and a liquid crystal display (LCD).

17. The patch of claim 16, wherein said visual indicator comprises a multicolored LED.

18. The patch of claim 1, said substrate further comprising:
a metal foil for electromagnetic interference shielding.

19. The patch of claim 1, further comprising:
means for automatic powering and activation of said patch upon either of opening of a package containing said patch and placement of said patch on a person's skin.

20. The patch of claim 1, wherein said heart condition comprises any of bradycardia, tachycardia, fibrillation, arrhythmia, normal heart function, premature contraction, late potentials, long QT syndrome, blocks and myocardial infarction.

21. The patch of claim 1, wherein said means for detecting a heart abnormality comprises means for performing signal averaging.

22. The patch of claim 1, wherein said means for detecting a heart abnormality comprises means for performing ST-segment analysis.

23. The disposable patch of claim 1, wherein said means for detecting a heart abnormality comprises means for performing QT-segment analysis.

24. The patch of claim 1, wherein said patch operates for about 24 hours.

25. The patch of claim 1, wherein said patch operates for about for 48 hours.

26. A patch for non-invasive detection of a heart condition, comprising:
means for adhering said patch is adhered to a person's chest, said person being evaluated for a possible heart abnormality;
at least two electrodes contacting said person's skin surface, said electrodes receiving a surface potential ECG signal;
an amplifier for amplifying said ECG signals from said electrodes;
a processor for performing analysis of said amplified ECG signals;
a memory;
a flexible substrate incorporating said amplifier, said processor, said electrodes, and said memory;
means for detecting a heart abnormality after analyzing said ECG signals by said processor and storing detection results in memory; and
means for transmitting heart detection results to an external device.

27. The patch of claim 26, further comprising:
a battery for powering said patch.

28. The patch of claim 26, wherein said processor analyzes said ECG signals for at least 90 seconds.

29. The disposable patch of claim 26, wherein said heart condition comprises any of bradycardia, tachycardia, fibrillation, a arrhythmia, normal heart function, premature contraction, late potentials, long QT syndrome, blocks, and myocardial infarction.

30. A method of non-invasive sensing of cardiac abnormalities, comprising the steps of:
adhering a patch to a person's chest, said patch comprising within an ECG amplifier, a processor, at least two electrodes for contacting said person's skin, and an indicator;
amplifying said ECG signal from said electrodes; and
producing an amplified ECG signal;
analyzing said amplified ECG signal with said processor;
detecting a heart condition by analysis of said processor; and
indicating said heart condition via said indicator.

31. The method of claim 30, wherein said heart condition is indicated after at least 90 seconds of placing said patch on said person.

32. The method of claim 30, wherein said analyzing is up to 24 hours.

33. The method of claim 30, wherein said analyzing continues for a period of up to 48 hours.

34. The method of claim 30, wherein said heart condition comprises any of arrhythmia, bradycardia, tachycardia, fibrillation, myocardial infarction, premature contraction, normal heart function, late potentials, blocks and long QT syndrome.

35. The method of claim 30, wherein said step of indicating of a heart condition is performed by a visual display means comprising any of an LED and an LCD.

36. The method of claim 30, wherein said step of indicating of a heart condition is performed by an audible means.

37. The method of claim 30, further comprising the steps of:
storing ECG data obtained by said disposable patch in a memory incorporated in said patch; and
transmitting said ECG date to an external device.

38. The method of claim 30, wherein said disposable patch is “C” shaped encompassing the person's breast area.

39. A method for non-invasive sensing of cardiac abnormalities, comprising the steps of:
adhering a patch to a person's chest, said patch comprising an ECG amplifier, a processor, at least two electrodes for contacting said person's skin, and a memory;
amplifying an ECG signal from said electrodes;
producing an amplified ECG signal;
analyzing said amplified ECG signal with said processor;
detecting a heart condition with said processor;
storing heart condition data in said memory; and
transmitting said heart condition data to an external device.

40. The method of claim 39, wherein said processor analyzes said ECG signals for at least 90 seconds.

41. A method for non-invasive detection of an ischemic heart condition, comprising the steps of:
adhering a patch non-invasively to a person's chest, said patch comprising an ECG amplifier, a processor, at least two electrodes for contacting said person's skin, and an indicator,
amplifying an ECG signal from said electrodes;
producing an amplified ECG signal;
analyzing an ST-segment of said amplified ECG signal with said processor;
detecting an ischemic heart condition from analysis of said ST-segment; and
indicating said ischemic heart condition via said indicator.

42. A patch for non-invasive detection of an ischemic heart condition, comprising:
means for adhering said patch to a person's chest,
at least two electrodes for said person's skin surface, said electrodes receiving a surface potential ECG signal;
an amplifier for amplifying said ECG signals from said electrodes;
a processor for performing analysis of said amplified ECG signals;
a flexible substrate incorporating said amplifier, said processor, and said electrodes; and
means for detecting an ischemic heart condition after analyzing an ST-segment of said ECG signals with said processor.

43. The patch of claim 42, further comprising:
a battery for powering said patch.

44. The patch of claim 42, wherein said processors analyzes said ST-segment of said ECG signals for at least 90 seconds.

45. A patch for non-invasive detection of a specific heart abnormality, comprising:
means for adhering said patch to a person's chest;
at least two electrodes for contacting said person's skin surface, said electrodes receiving a surface potential ECG signal;
an amplifier for amplifying said ECG signals from said electrodes;
a processor for performing analysis of said amplified ECG signals;
a flexible substrate comprising said amplifier, said processor, and said electrodes; and
a processor implemented algorithm for detecting a specific heart abnormality after analyzing said ECG signals with said processor.

46. The patch of claim 45, furthering comprising:
a battery for powering said patch.

47. The patch of claim 45, wherein said processor analyzes said ECG signals for at least 90 seconds.

48. A patch for non-invasive detection of a heart disease, comprising:
a “C” shaped substrate for encompassing a person's left breast said patch comprising a vertical segment along said person's sternum, an upper segment above said person's breast, and a lower segment below said person's breast; and
means for adhering said patch to said person's chest.

49. The patch of claim 48, further comprising:
at least five electrodes for contacting said person's skin, said electrodes receiving a surface potential ECG signal;
an amplifier for amplifying said ECG signals from said electrodes;
a processor for performing analysis of said amplified ECG signals;
said substrate comprising said amplifier, said processor, and said electrodes; and
a processor implemented algorithm for detecting a heart disease after analyzing said ECG signals with said processor.

50. The patch of claim 48, further comprising:
a battery for powering said disposable patch.

51. The patch of claim 48, wherein said processor analyzes said ECG signals for at least 90 seconds.
